SEQUENCE LISTING (77 sequences)

SEQ ID NO: 1 - DNA, 712 bp, Leishmania donovani
ggttggtgtaaaatagggccgggtggtggtcgggaatgggctaccctgggctggattggcctggacccctgggtctggaggcttgatttcgatctggttgggattgtctttattttgctggctttgggcttgagtttagtgactaaacggggctagggctttgattctggtgtttggggtggtgctttagggtatgcgtgggcatctgttttgtgcttattatttataattatgactgggtgtacagtcactttagttcgctccgcatggaggccgatgggaattggatgaccgtattcggtggcggggtaatgttagttagcctttctttagccactatagaataaagggaatatctaattccggcatagctataggatgtggtagggtaagcgtgacatatagtcaagttatattgtgagtttacgaatcaattggacttagcgcctactcatacattatatactcttgaatgtgaagtttttgatctgtttggttatgttatgatcgaggctgacacaatttgatggcctgtgtaaatatatcttaccttaaggtggcttctttgaaatgcaatttgaattaatttggggatgggttgctggaggtgttgggacgtgttggatggttgggaattggtggaaaatcccgaaaaatggtcgaaaaatgccaaaaatcccaaacttttctggtcctccgggtaggggcgttc

SEQ ID NO: 2 - DNA, 54 bp, Artificial Sequence
  synthetic oligonucleotide
ctacccggaggaccagaaaagtttgggctacccggaggaccagaaaagtttggg

SEQ ID NO: 3 - DNA, 31 bp, Artificial Sequence
  synthetic oligonucleotide
gatcgaggctgacacaatttgatggcctgtg

SEQ ID NO: 4 - DNA, 34 bp, Artificial Sequence
  synthetic oligonucleotide
tggcctgtgtaaatatatcttaccttaaggtggc

SEQ ID NO: 5 - DNA, 31 bp, Artificial Sequence
  synthetic oligonucleotide
tctgtttggttatgttatgatcgaggctgac

SEQ ID NO: 6 - DNA, 28 bp, Artificial Sequence
  synthetic oligonucleotide
ctacccggaggaccagaaaagtttggga

SEQ ID NO: 7 - DNA, 30 bp, Artificial Sequence
  synthetic oligonucleotide
ttcgggattttccaccaattcccaaccatc

SEQ ID NO: 8 - DNA, 30 bp, Artificial Sequence
  synthetic oligonucleotide
accaattcccaaccatccaacacgtcccaa

SEQ ID NO: 9 - DNA, 45 bp, Artificial Sequence
  synthetic oligonucleotide
ctttgaaatgcaatttgaattaatttggggatgggttgctggagg

SEQ ID NO: 10 - DNA, 30 bp, Artificial Sequence
  synthetic oligonucleotide
ccatagcgctttagaatagttcgactccga

SEQ ID NO: 11 - DNA, 29 bp, Artificial Sequence
  synthetic oligonucleotide
atagcgctttagaatagttcgactccgaa

SEQ ID NO: 12 - DNA, 30 bp, Artificial Sequence
  synthetic oligonucleotide
atcggtatagatattactactacacacagc

SEQ ID NO: 13 - DNA, 32 bp, Artificial Sequence
  synthetic oligonucleotide
gcagtatgcgccaacctataaatatatcggta

SEQ ID NO: 14 - DNA, 45 bp, Artificial Sequence
  synthetic oligonucleotide
ataactgacattactcgtacactataagtattatgtttaatatat

SEQ ID NO: 15 - DNA, 722 bp, Leishmania braziliensis
  misc_feature (600)..(600): n is a, c, g, or t
ggggaggcgacatccgggccctgcagccccacttaccgtggcgcgggaatgttaaagggcccaggccaaaaattatccctccgggccgggataatgggaaggggtaaataaataggtaattaaaattattaaattagccatctgccagtaaaattaggtaattgtaaaaaatttagggaggaataccattatcgatacgctaaggtagtacctatgggaaatgcattaggggggtataggtccaataaggtggataatataaggggaataataacgtgtacctaatatccgggggaattacctatgttaaccctataatactctaactatacaacgctaattatactatattactatgctactaatgttagaacctatttagtaacaatattagattcaatacatagctttattaataaactaatttatatacatttttatatttataaatcgtgttactactattgatacaacattaaaatatttttattaacgtaataatcttttgcgcagagttattatgttgtcaatgaataatttaatttaaatatcataaaatttgatgaaaatgtactccccgacatgcctctgggtaggggngttctgcgaaaaccgaaaaatggcatacagaaaccccgttcaaaaaatgcccgaaaatcgctatttttggcctccccgtgcacaattaggggttggtgtaatatagtgggccgcgcaccccg

SEQ ID NO: 16 - DNA, 31 bp, Artificial Sequence
  synthetic oligonucleotide
tgcgcagagttattatgttgtcaatgaataa

SEQ ID NO: 17 - DNA, 30 bp, Artificial Sequence
  synthetic oligonucleotide
gatgaaaatgtactccccgacatgcctctg

SEQ ID NO: 18 - DNA, 30 bp, Artificial Sequence
  synthetic oligonucleotide
aatttgatgaaaatgtactccccgacatgc

SEQ ID NO: 19 - DNA, 32 bp, Artificial Sequence
  synthetic oligonucleotide
tgagacggggtttctgtatgccatttttcggt

SEQ ID NO: 20 - DNA, 32 bp, Artificial Sequence
  synthetic oligonucleotide
ctaattgtgcacggggaggccaaaaatagcga

SEQ ID NO: 21 - DNA, 29 bp, Artificial Sequence
  synthetic oligonucleotide
aacccctaattgtgcacggggaggccaaa

SEQ ID NO: 22 - DNA, 1423 bp, Trypanosoma cruzi
cacaatctcctaatccatacgatttgtactctaacgcatttcaatattcacaatcacagtctaaactgtaacaaaactatctatataccaatctatcaaattaaatcctataaccaactggcactatattacaccaaccccaatcgaaccccacctccccgtaaaatccgccaaatccataaaataatgtacgggggagatgcatgaatttccgccccaaaagttgaacgcccctcccaaaatcaaagtttgcgtatttccgaaccccattttatgataaataacaaacaccaaaccataccaaccacccatagttatacactacccctcatacattatatcaaaacatatacaacagataataaacataatcattataactcaatctcacactactatacgaacacacaactatccaactccagatgcacctcatatccaattcaactacacaaattcataaatacatattcatctatattacaccaaccccaatcgaaccccacctccccgaaaattccgccaaatccataaaataatgtacgggggagatgcatgaattctggccccaaaagttgaacgcccctcccaaaacagagtttccggaaattccaggataccaattttaaccactaacagcctacatcaaaccacaacgcaccatattatataaacatatactgtaacatagaacacatcattaactatcaaacttatacactactacaaaccactacattatcactacccatctatcaaacatcatatcacaatcataaagaaacaactaatctaaaactatattccattattaccccataacccacagcccgtaatattacaccaaccccaatcgaaccccacctccccgtaaaatccgccaaatccataaaataatgtacggggagatgcatgaatttccgccccaaaagttgaacgcccctcccaaaaccagaaatcccagaaattttatagcctacatttcgtaactaactacctacgccaaccccacctccaacagtaagacattaatatacataacaacaatatcaagtatacaataccatactattactatcacctctcactatattcaaatccacacgtcaacattcatagtactataccaatcataaacaagataataaaactataatagccttatcacccttaccacccacagcctatattacaccaaccccaatcgaaccccacctccccgtaaaatcggtaaaatccccaaaataatgtacgggggagatgcatgattctgggccccaaaagttgaacgcccctcccaaaaccatattttccagtttcttgcagacctatttcataccacccatcacaataaacaatcaacattatatatattattaaactataaactatatacattatcaatcaccaaatctacataact

SEQ ID NO: 23 - DNA, 30 bp, Artificial Sequence
  synthetic oligonucleotide
agccttatcacccttaccacccacagccta

SEQ ID NO: 24 - DNA, 30 bp, Artificial Sequence
  synthetic oligonucleotide
cagcctatattacaccaaccccaatcgaac

SEQ ID NO: 25 - DNA, 31 bp, Artificial Sequence
  synthetic oligonucleotide
acccttaccacccacagcctatattacacca

SEQ ID NO: 26 - DNA, 30 bp, Artificial Sequence
  synthetic oligonucleotide
gcgttcaacttttggggcccagaatcatgc

SEQ ID NO: 27 - DNA, 30 bp, Artificial Sequence
  synthetic oligonucleotide
aacttttggggcccagaatcatgcatctcc

SEQ ID NO: 28 - DNA, 32 bp, Artificial Sequence
  synthetic oligonucleotide
ctgcaagaaactggaaaatatggttttgggag

SEQ ID NO: 29 - DNA, 45 bp, Artificial Sequence
  synthetic oligonucleotide
caacttttggggcggaaattcatgcatctcccccgtacattattt

SEQ ID NO: 30 - DNA, 45 bp, Artificial Sequence
  synthetic oligonucleotide
tgaattgtgatgggatactgtgagtgtcttgttatacttttgtgt

SEQ ID NO: 31 - DNA, 804 bp, Leishmania infantum
ggggttggtgtaaaatagggccgggtggtggccggaaatggctcccctgggctggattgggttttcctgagctggattgggctgaaaccctggggttgggggcttggttttgggtgggctagcgtttttggtgttttaatggttcgggtgcctttgatgtgggtgttggttgtgtggctttagtgggttggagcctggtggaggcttgtgccttggtggattagtctggtggggtgcttcggtgttactctacgggtgtctttgatgatgctggggtgggttgactttaatgttggggttagccgatggtggtcttgggttgaccttaggtggtgattctctttaatattgatattaaaggtaagtgcttggacatatgatatagccatagcgctttagaatagttcgactccgaagacccagtctgaggtagtgttaagtatacattaatctagtatataatttagcatataataactgacattgctcgtacactataagtattatgtttaatatattgctgtagtatgtatttgtgtgctgtgtgtagtagtaatatctataccgatatatttataggttggcgcatactgcagtgaattgaaaattaatgaattggggtcaggctgtggggggtgtcgtaaattctggaaaatgatggaaaatggccaaaaatggggggaaattccaaacttttctggtcctccgggtaggggcgttctgcaaaatcggaaaaatgggtgcagaaatcccgttcaaaaatcggccaaaaatgccaaaaatcggctccggggcgggaaactg

SEQ ID NO: 32 - DNA, 34 bp, Artificial Sequence
  synthetic oligonucleotide
tagccatagcgctttagaatagttcgrctccgaa

SEQ ID NO: 33 - DNA, 35 bp, Artificial Sequence
  synthetic oligonucleotide
tatagccatagcgctttagaatagttcgrctccga

SEQ ID NO: 34 - DNA, 28 bp, Artificial Sequence
  synthetic oligonucleotide
tatagccatagcgctttagaatagttcg

SEQ ID NO: 35 - DNA, 34 bp, Artificial Sequence
  synthetic oligonucleotide
trctcgtacactataagtattatgtttaatatat

SEQ ID NO: 36 - DNA, 30 bp, Artificial Sequence
  synthetic oligonucleotide
ctgacattrctcgtacactatagtattatg

SEQ ID NO: 37 - DNA, 44 bp, Artificial Sequence
  synthetic oligonucleotide
taataactacattactcgtacactataagtattatgtttaatat

SEQ ID NO: 38 - DNA, 33 bp, Artificial Sequence
  synthetic oligonucleotide
tagtaatatctrtaccgatatatttrtaggttg

SEQ ID NO: 39 - DNA, 33 bp, Artificial Sequence
  synthetic oligonucleotide
caacctayaaatatatcggtayagatattacta

SEQ ID NO: 40 - DNA, 33 bp, Artificial Sequence
  synthetic oligonucleotide
catactgcagtgaattgraaattaatraattgg

SEQ ID NO: 41 - DNA, 33 bp, Artificial Sequence
  synthetic oligonucleotide
ccaattyattaatttycaattcactgcagtatg

SEQ ID NO: 42 - DNA, 28 bp, Artificial Sequence
  synthetic oligonucleotide
ccctacccggaggaccagaaaagtttgg

SEQ ID NO: 43 - DNA, 40 bp, Artificial Sequence
  synthetic oligonucleotide
ccaaacttttctggtcctccgggtaggggcgttctgcaaa

SEQ ID NO: 44 - DNA, 28 bp, Artificial Sequence
  synthetic oligonucleotide
gaaaaatgggtccagaaatgccgttcaa

SEQ ID NO: 45 - DNA, 28 bp, Artificial Sequence
  synthetic oligonucleotide
ttgaacgggatttctgcacccatttttc

SEQ ID NO: 46 - DNA, 30 bp, Artificial Sequence
  synthetic oligonucleotide
aaactgggggttggtgtaaaatagggccgg

SEQ ID NO: 47 - DNA, 30 bp, Artificial Sequence
  synthetic oligonucleotide
ccggccctattttacaccaacccccagttt

SEQ ID NO: 48 - DNA, 29 bp, Artificial Sequence
  synthetic oligonucleotide
ggaaactgggggttggtgtaaaatagggc

SEQ ID NO: 49 - DNA, 29 bp, Artificial Sequence
  synthetic oligonucleotide
gccctattttacaccaacccccagtttcc

SEQ ID NO: 50 - DNA, 28 bp, Artificial Sequence
  synthetic oligonucleotide
cacgcgcgctcaaccatacaaattgtcg

SEQ ID NO: 51 - DNA, 30 bp, Artificial Sequence
  synthetic oligonucleotide
gcgtcttgtcagtatggacttattgccgtt

SEQ ID NO: 52 - DNA, 44 bp, Artificial Sequence
  synthetic oligonucleotide
atggcagatggtcgcacccaacgctccggtaaaacacgcccagc

SEQ ID NO: 53 - DNA, 30 bp, Artificial Sequence
  synthetic oligonucleotide
taatatacacttgcacgcgttacgagtgtg

SEQ ID NO: 54 - DNA, 29 bp, Artificial Sequence
  synthetic oligonucleotide
atataaaccacacgacgaaatatgcgcat

SEQ ID NO: 55 - DNA, 45 bp, Artificial Sequence
  synthetic oligonucleotide
tgtgtagaaaaatgtaattgcggcttgacttcgcgtgtgcacata

SEQ ID NO: 56 - DNA, 27 bp, Artificial Sequence
  synthetic oligonucleotide
aatgacggtgcggcgaccttaactgtc

SEQ ID NO: 57 - DNA, 29 bp, Artificial Sequence
  synthetic oligonucleotide
cggcgaccttaactgtctgcaacaacact

SEQ ID NO: 58 - DNA, 29 bp, Artificial Sequence
  synthetic oligonucleotide
gcgaccttaactgtctgcaacaacactgc

SEQ ID NO: 59 - DNA, 30 bp, Artificial Sequence
  synthetic oligonucleotide
ccatacaaaacacccttcacggattgcatt

SEQ ID NO: 60 - DNA, 29 bp, Artificial Sequence
  synthetic oligonucleotide
ccccatacaaaacacccttcacggattgc

SEQ ID NO: 61 - DNA, 45 bp, Artificial Sequence
  synthetic oligonucleotide
cggccatttttcattttggacacttaagtttttgtgcggtaaagt

SEQ ID NO: 62 - DNA, 45 bp, Artificial Sequence
  synthetic oligonucleotide
agtttttgtgcggtaaagtgagttttggtttgtgagactgtttgt

SEQ ID NO: 63 - DNA, 30 bp, Artificial Sequence
  synthetic oligonucleotide
tacatgcatgcgagaggaacaactgtgatg

SEQ ID NO: 64 - DNA, 30 bp, Artificial Sequence
  synthetic oligonucleotide
tatattgcgaagtcaagcggcaagtaggtt

SEQ ID NO: 65 - DNA, 45 bp, Artificial Sequence
  synthetic oligonucleotide
tgttgcgtcctcttcgcaccaagatttgcacgcacatgtgtgcat

SEQ ID NO: 66 - DNA, 30 bp, Artificial Sequence
  synthetic oligonucleotide
gtataggcttttcccacatacacagcaaac

SEQ ID NO: 67 - DNA, 29 bp, Artificial Sequence
  synthetic oligonucleotide
gacgttgtagaacgcaccgcctatacaca

SEQ ID NO: 68 - DNA, 29 bp, Artificial Sequence
  synthetic oligonucleotide
ttatgtgagccgttatccacgcgcacgca

SEQ ID NO: 69 - DNA, 44 bp, Artificial Sequence
  synthetic oligonucleotide
aaaatagacggcgtttcggtttttggcgggagggagagagaggg

SEQ ID NO: 70 - DNA, 30 bp, Artificial Sequence
  synthetic oligonucleotide
aggcctattctttcgataggcgcctttccc

SEQ ID NO: 71 - DNA, 30 bp, Artificial Sequence
  synthetic oligonucleotide
ctgcttttgtatatacgcggttgtgcgcta

SEQ ID NO: 72 - DNA, 44 bp, Artificial Sequence
  synthetic oligonucleotide
cagcaaagtttttgtactcaaaaacaacatttgcagtaaacaaa

SEQ ID NO: 73 - DNA, 28 bp, Artificial Sequence
  synthetic oligonucleotide
aaacatacaactccggggaggcttgtgt

SEQ ID NO: 74 - DNA, 30 bp, Artificial Sequence
  synthetic oligonucleotide
gatcggcctttttctactgtatgttgtgtc

SEQ ID NO: 75 - DNA, 30 bp, Artificial Sequence
  synthetic oligonucleotide
atacgcgatatgttaacgtcgatcggcctt

SEQ ID NO: 76 - DNA, 43 bp, Artificial Sequence
  synthetic oligonucleotide
tctagcaagcctttcccacagatacgcaatacaatctatatat

SEQ ID NO: 77 - DNA, 46 bp, Artificial Sequence
  synthetic oligonucleotide
gtaggggggttctgcgaaaaccgaaaaatggcatacagaaaccccg

(19) United States
(12) Patent Application Publication
(10) Pub. No.: US 2016/0130669 A1
(43) Pub. Date: May 12, 2016

(54) RECOMBINASE POLYMERASE AMPLIFICATION (RPA) METHOD FOR LEISHMANIA SPP. AND TRYPANOSOMA CRUZI

(71) Applicants: Alejandro Castellanos, Galveston, TX (US); Bruno Travi, Galveston, TX (US); Omar Saldarriaga, Galveston, TX (US); Peter Melby, Galveston, TX (US)

(72) Inventors: Alejandro Castellanos, Galveston, TX (US); Bruno Travi, Galveston, TX (US); Omar Saldarriaga, Galveston, TX (US); Peter Melby, Galveston, TX (US)

(73) Assignee: The Board of Regents Of The University Of Texas System, Austin, TX (US)

(21) Appl. No.: 14/993,407

(22) Filed: Jan. 12, 2016

Related U.S. Application Data
(63) Continuation-in-part of application No. PCT/US2014/046448, filed on Jul. 12, 2014.
(60) Provisional application No. 61/845,884, filed on Jul. 12, 2013.

(51) Int. Cl. C12Q 1/68

(52) U.S. Cl. CPC C12Q 1/6893; C12Q 1/6844; C12Q 2600/16

(57) ABSTRACT

Certain embodiments are directed to a sensitive isothermal amplification test based on the recombinase polymerase amplification (RPA) method that is capable of detecting <2 parasites in a biological sample.

### PRIORITY

This application claims priority to and is a continuation-in-part of International application serial number PCT/US2014/046448 filed Jul. 12, 2014. Priority is also claimed to U.S. Provisional Patent Application Ser. No. 61/845,884 filed Jul. 12, 2013. Each of the above referenced applications is incorporated herein by reference in its entirety.

### BACKGROUND

Leishmaniasis, caused by the protozoan Leishmania, is a disease with significant global impact affecting more than 88 countries in the world. Cutaneous leishmaniasis (CL) is characterized by chronic skin ulcers that can impact the individual's functional status, lead to expensive and untimely treatment, and result in disfiguring scarring. Collectively, there are 10-20 million cases of leishmaniasis worldwide. Leishmaniasis causes a spectrum of diseases that include localized cutaneous leishmaniasis (LCL), destructive nasal and oropharyngeal lesions of mucosal leishmaniasis (ML). LCL in the New World is most commonly caused by species of the Viannia subgenus (L. (V.) braziliensis, L. (V.) panamensis, L. (V.) guyanensis, L. (V.) peruviana) and to a lesser extent by species of the Leishmania subgenus (L. (V.) mexicana, L. (V.) amazonensis). ML is typically caused by L. braziliensis and less frequently by L. (V.) panamensis or L. (V.) guyanensis.

A major challenge in the diagnosis of leishmaniasis is that the disease occurs in remote and resource-limited areas of the world. Current parasitological diagnosis must be performed in hospital settings not accessible to the majority of patients. This also could be true during military field operations and training exercises where sophisticated laboratory equipment and medical personnel are scarce or not available. For CL or ML, scrapings of cutaneous (dermal) or mucosal tissues or punch biopsies of the lesions are necessary and the diagnostic sensitivity by histopathology, microscopy of smears or culture could be unacceptably low (40-70%). Antibody-based tests are not useful to diagnose any clinical form of cutaneous or mucosal leishmaniasis. PCR is a highly sensitive detection method for diagnosis of CL but the costs, personnel training and need of sophisticated equipment and laboratories has precluded its implementation in resource-poor settings or in sites with deployed military personnel.

Novel methods to detect infectious diseases at the POC are urgently needed and this is also true for leishmaniasis. These methods need to be fast, inexpensive, specific and sensitive, not require expensive equipment prone to malfunction.

There is a need for additional methods for diagnosing parasite infections.

### SUMMARY

Early diagnosis of visceral leishmaniasis (VL) can play a pivotal role in diminishing disease burden and mortality. To assist in early diagnosis affordable field-adapted diagnostic tools and methods are described herein.

The inventors have developed a sensitive isothermal amplification test based on the recombinase polymerase amplification (RPA) method that is capable of detecting medically important species of Trypanosomatida in a biological sample. Trypanosomatids are a group of kinetoplastid protozoa distinguished by having only a single flagellum. A few genera of Trypanosomatida have life-cycles involving a secondary host, which may be a vertebrate, invertebrate or plant. These include several species that cause major diseases in humans, e.g., Leishmania and Trypanosoma. In certain aspects oligonucleotide primers are designed to amplify Leishmania or Trypanosoma nucleic acids. In certain aspects primers are directed to kinetoplast DNA (kDNA). As used herein a kinetoplast is a network of circular DNA (called kDNA) inside a large mitochondrion that contains many copies of the mitochondrial genome. Kinetoplasts are only found in protozoa of the class Kinetoplastida. A kinetoplast is usually adjacent to the organism's flagellar basal body, suggesting that it is tightly bound to the cytoskeleton. In certain aspects primers are designed so that they do not cross-amplify nucleic acids (e.g., kDNA) of other Leishmania or Trypanosoma. By using these primers one can identify a subject, e.g., humans or dogs, infected with a particular parasite (e.g., L. infantum) and rule out the possibility of detecting related protozoan parasites having an overlapping host range.

Recombinase Polymerase Amplification (RPA) is a single tube, isothermal alternative to the Polymerase Chain Reaction (PCR). By adding a reverse transcriptase enzyme to an RPA reaction it can detect RNA as well as DNA, without the need for a separate step to produce cDNA. Because it is isothermal, RPA reactions need much simpler equipment than PCR. RPA reactions can in theory be run quickly simply by holding a tube. This makes RPA an excellent candidate for developing low-cost, rapid, point-of-care molecular tests. In contrast to conventional PCR, which requires expensive equipment, RPA is performed at constant temperature without sophisticated equipment.

In certain aspects a kit and/or a reaction mixture as described herein comprises one or more of a recombinase, a single-stranded DNA-binding protein (SSB), and/or strand-displacing polymerase. In a further aspect a kit and/or a reaction mixtures can further comprise an exonuclease (e.g., exo III), an endonuclease (endo IV) or a reverse transcriptase. In addition to the enzyme-reaction mix a kit can include rehydration buffers, reaction buffers, primers, labels, detection reagents and the like. As with PCR, all forms of RPA reactions can be multiplexed by the addition of further primer/probe pairs, allowing the detection of multiple analytes or an internal control in the same tube.

Isothermal amplification by RPA, as compared to loop mediated isothermal amplification (LAMP) or other isothermal amplification methods is less complex and can be adapted easily to lateral flow detection. Recent publications have confirmed the applicability of this technology to detect viral and bacterial infections (Euler et al., 2013). The methods described herein use RPA to detect parasites under isothermal amplification conditions (FIG. 1).

Certain embodiments are directed to amplification primers or RPA primers that specifically amplify particular parasites to produce parasite specific amplicons. In certain aspects the primers, probes, and amplicons are Leishmania donovani, Leishmania chagasi, and/or Leishmania infantum specific. In a further aspect, oligonucleotide probes are designed to detect these parasite specific amplicons. The probes are specifically designed to target unique DNA sequences between and amplified by the two primers. The primer and probe combinations dictate the specificity (which DNA sequence will be detected) to the test. In certain aspects, the Leishmania donovani, Leishmania chagasi, and/or Leishmania infantum specific amplification primers include oligonucleotides having a sequence 90, 95, 98 or 100% identical, including all ranges and values there between, to SEQ ID NO:3, 4, 5, 6, 7, 8, 10, 11, 12, or 13. In certain aspects, the amplification primers include oligonucleotides having 25, 26, 27, 28, 29, 30, 31, 32, 33, 34, 35, 36, 37, 38, 39, 40, 41, 42, 43, 44, or 45 consecutive nucleotides, including all ranges there between, of SEQ ID NO: 3, 4, 5, 6, 7, 8, 10, 11, 12, or 13. Certain aspects are directed to amplification primer pairs comprising SEQ ID NOs:3/6, 3/7, 3/8, 4/6, 4/7, 4/8, 5/6, 5/7, 5/8, 10/12, 10/13, 11/12, or 11/13. RPA probes can include oligonucleotides having a nucleotide sequence of SEQ ID NO:9 or 14. In certain aspects SEQ ID NO:14 can include a tetrahydrofuran (THF) spacer in place of the guanosine at position 28.

In certain aspects the primers, probes, and amplicons are Leishmania braziliensis specific. In a further aspect, oligonucleotide probes are designed to detect these parasite specific amplicons. In certain aspects, the Leishmania braziliensis amplification primers include oligonucleotides having a sequence 70, 75, 80, 85, 90, 95, 98 or 100% identical, including all ranges and values there between, to SEQ ID NO:16, 17, 18, 19, 20, and/or 21. In certain aspects, the amplification primers include oligonucleotides having 25, 26, 27, 28, 29, 30, 31, 32, 33, 34, 35, 36, 37, 38, 39, 40, 41, 42, 43, 44, or 45 consecutive nucleotides, including all ranges there between, of SEQ ID NO: 16, 17, 18, 19, 20, and/or 21. Certain aspects are directed to amplification primer pairs comprising SEQ ID NOs: 16/19, 16/20, 16/21, 17/19, 17/20, 17/21, 18/19, 18/20, or 18/21. RPA probes can include oligonucleotides having a nucleotide sequence of SEQ ID NO:77.

In certain aspects the primers, probes, and amplicons are Trypanosome cruzi or Trypanosome rangeli specific. In a further aspect, oligonucleotide probes are designed to detect these parasite specific amplicons. In certain aspects, the Trypanosoma cruzi amplification primers include oligonucleotides having a sequence 90, 95, 98 or 100% identical, including all ranges and values there between, to SEQ ID NO:23, 24, 25, 26, 27, and/or 28. In certain aspects, the amplification primers include oligonucleotides having 25, 26, 27, 28, 29, 30, 31, 32, 33, 34, 35, 36, 37, 38, 39, 40, 41, 42, 43, 44, or 45 consecutive nucleotides, including all ranges there between, of SEQ ID NO: 23, 24, 25, 26, 27, and/or 28. Certain aspects are directed to amplification primer pairs comprising SEQ ID NOs: 23/26, 23/27, 23/28, 24/26, 24/27, 24/28, 25/26, 25/27, or 25/28. RPA probes can include oligonucleotides having a nucleotide sequence of SEQ ID NO:29 for T. cruzi or SEQ ID NO:30 for T. rangeli.

In certain aspects the primers, probes, and amplicons are Leishmania chagasi specific. In a further aspect, oligonucleotide probes are designed to detect these parasite specific amplicons. In certain aspects, the Leishmania chagasi amplification primers include oligonucleotides having a sequence 90, 95, 98 or 100% identical, including all ranges and values there between, to SEQ ID NO:32, 33, 34, 35, 36, 37, 38, 39, 40, and/or 41. In certain aspects, the amplification primers include oligonucleotides having 25, 26, 27, 28, 29, 30, 31, 32, 33, 34, 35, 36, 37, 38, 39, 40, 41, 42, 43, 44, or 45 consecutive nucleotides, including all ranges there between, of SEQ ID NO: 32, 33, 34, 35, 36, 37, 38, 39, 40, and/or 41. Certain aspects are directed to amplification primer pairs comprising SEQ ID NOs:32/39, 32/40, 32/41, 32/42, 33/39, 33/40, 33/41, 33/42, 34/39, 34/40, 34/41, 34/42, 35/39, 35/40, 35/41, 35/42, 36/39, 36/40, 36/41, 36/42, 38/39, 38/40, 38/41, or 38/42. RPA probes can include oligonucleotides having a nucleotide sequence of SEQ ID NO:37.

In certain aspects the primers, probes, and amplicons are Trypanosoma cruzi specific. In a further aspect, oligonucleotide probes are designed to detect these parasite specific amplicons. In certain aspects primers and probes can be designed to amplify subtelomeric regions of T. cruzi. In certain aspects, the Trypanosoma cruzi amplification primers include oligonucleotides having a sequence 90, 95, 98 or 100% identical, including all ranges and values there between, to SEQ ID NO:50 and/or 51. In certain aspects, the amplification primers include oligonucleotides having 25, 26, 27, 28, 29, 30, 31, 32, 33, 34, 35, 36, 37, 38, 39, 40, 41, 42, 43, 44, or 45 consecutive nucleotides, including all ranges there between, of SEQ ID NO:50 and/or 51. Certain aspects are directed to amplification primer pairs comprising SEQ ID NOs:50/51. RPA probes can include oligonucleotides having a nucleotide sequence of SEQ ID NO:52.

Trypanosoma cruzi. In certain aspects the primers, probes, and amplicons are Trypanosoma cruzi specific. In a further aspect, oligonucleotide probes are designed to detect these parasite specific amplicons. In certain aspects primers and probes can be designed to amplify ITS1 region of T. cruzi. In certain aspects, the Trypanosoma cruzi amplification primers include oligonucleotides having a sequence 90, 95, 98 or 100% identical, including all ranges and values there between, to SEQ ID NO:53 and/or 54. In certain aspects, the amplification primers include oligonucleotides having 25, 26, 27, 28, 29, 30, 31, 32, 33, 34, 35, 36, 37, 38, 39, 40, 41, 42, 43, 44, or 45 consecutive nucleotides, including all ranges there between, of SEQ ID NO:53 and/or 54. Certain aspects are directed to amplification primer pairs comprising SEQ ID NOs:53/54. RPA probes can include oligonucleotides having a nucleotide sequence of SEQ ID NO:55.

In certain aspects the primers, probes, and amplicons are Trypanosoma rangeli specific. In a further aspect, oligonucleotide probes are designed to detect these parasite specific amplicons. In certain aspects primers and probes can be designed to amplify subtelomeric regions of T. rangeli. In certain aspects, the Trypanosoma rangeli amplification primers include oligonucleotides having a sequence 90, 95, 98 or 100% identical, including all ranges and values there between, to SEQ ID NO:56, 57, 58, 59, and/or 60. In certain aspects, the amplification primers include oligonucleotides having 25, 26, 27, 28, 29, 30, 31, 32, 33, 34, 35, 36, 37, 38, 39, 40, 41, 42, 43, 44, or 45 consecutive nucleotides, including all ranges there between, of SEQ ID NO: 56, 57, 58, 59, and/or 60. Certain aspects are directed to amplification primer pairs comprising SEQ ID NOs: 56/59, 57/59, 58/59, 56/60, 57/60, and/or 58/60. RPA probes can include oligonucleotides having a nucleotide sequence of SEQ ID NO:61 or 62.

In certain aspects the primers, probes, and amplicons are Trypanosoma rangeli specific. In a further aspect, oligonucleotide probes are designed to detect these parasite specific amplicons. In certain aspects primers and probes can be designed to amplify ITS1 region of Trypanosoma rangeli. In certain aspects, the Trypanosoma rangeli amplification primers include oligonucleotides having a sequence 90, 95, 98 or 100% identical, including all ranges and values there between, to SEQ ID NO:63 or 64. In certain aspects, the amplification primers include oligonucleotides having 25, 26, 27, 28, 29, 30, 31, 32, 33, 34, 35, 36, 37, 38, 39, 40, 41, 42, 43, 44, or 45 consecutive nucleotides, including all ranges there between, of SEQ ID NO:63 or 64. Certain aspects are directed to amplification primer pairs comprising SEQ ID NOs:63/64. RPA probes can include oligonucleotides having a nucleotide sequence of SEQ ID NO:65.

Leishmania major. In certain aspects the primers, probes, and amplicons are Leishmania major specific. In a further aspect, oligonucleotide probes are designed to detect these parasite specific amplicons. In certain aspects primers and probes can be designed to amplify ITS1 intergenic region of Leishmania major. In certain aspects, the Leishmania major amplification primers include oligonucleotides having a sequence 90, 95, 98 or 100% identical, including all ranges and values there between, to SEQ ID NO:66, 67, or 68. In certain aspects, the amplification primers include oligonucleotides having 25, 26, 27, 28, 29, 30, 31, 32, 33, 34, 35, 36, 37, 38, 39, 40, 41, 42, 43, 44, or 45 consecutive nucleotides, including all ranges there between, of SEQ ID NO:66, 67, or 68. Certain aspects are directed to amplification primer pairs comprising SEQ ID NOs:66/68 or 67/68. RPA probes can include oligonucleotides having a nucleotide sequence of SEQ ID NO:69.

In certain aspects the primers, probes, and amplicons are Leishmania mexicana or Leishmania amazonensis specific. In a further aspect, oligonucleotide probes are designed to detect these parasite specific amplicons. In certain aspects primers and probes can be designed to amplify ITS1 region of Leishmania mexicana or Leishmania amazonensis. In certain aspects, the Leishmania mexicana or Leishmania amazonensis amplification primers include oligonucleotides having a sequence 90, 95, 98 or 100% identical, including all ranges and values there between, to SEQ ID NO:70 or 71. In certain aspects, the amplification primers include oligonucleotides having 25, 26, 27, 28, 29, 30, 31, 32, 33, 34, 35, 36, 37, 38, 39, 40, 41, 42, 43, 44, or 45 consecutive nucleotides, including all ranges there between, of SEQ ID NO:70 or 71. Certain aspects are directed to amplification primer pairs comprising SEQ ID NOs:70/71. RPA probes can include oligonucleotides having a nucleotide sequence of SEQ ID NO:72.

In certain aspects the primers, probes, and amplicons are Leishmania braziliensis, Leishmania panamensis, Leishmania guyanensis, and/or Leishmania peruviana specific. In a further aspect, oligonucleotide probes are designed to detect these parasite specific amplicons. In certain aspects primers and probes can be designed to amplify ITS1 regions of Leishmania braziliensis, Leishmania panamensis, Leishmania guyanensis, and/or Leishmania peruviana. In certain aspects, the Leishmania braziliensis, Leishmania panamensis, Leishmania guyanensis, and/or Leishmania peruviana amplification primers include oligonucleotides having a sequence 90, 95, 98 or 100% identical, including all ranges and values there between, to SEQ ID NO:73, 74, or 75. In certain aspects, the amplification primers include oligonucleotides having 25, 26, 27, 28, 29, 30, 31, 32, 33, 34, 35, 36, 37, 38, 39, 40, 41, 42, 43, 44, or 45 consecutive nucleotides, including all ranges there between, of SEQ ID NO:73, 74, or 75. Certain aspects are directed to amplification primer pairs comprising SEQ ID NOs:73/75 or 74/75. RPA probes can include oligonucleotides having a nucleotide sequence of SEQ ID NO:76.

Certain embodiments are directed to oligonucleotides having a sequence 90, 95, 98 or 100% identical, including all ranges and values there between, to SEQ ID NO:2, 3, 4, 5, 6, 7, 8, 16, 17, 18, 19, 20, 21, 23, 24, 25, 26, 27, 28, 32, 33, 34, 35, 36, 37, 38, 39, 40, 41, 42, 43, 44, 45, 46, 47, 48, or 49-77.

Certain embodiments are directed to a parasite detection kit comprising at least one nucleic acid amplification primer pair and a nucleic acid probe for detecting amplified Leishmania or Trypanosoma DNA segments. In certain aspects the amplification primer can be (a) capable of amplifying Trypanosome cruzi DNA having a nucleotide sequence of (i) SEQ ID NO: 23 and one or more of SEQ ID NO:26, SEQ ID NO:27, or SEQ ID NO:28; (ii) SEQ ID NO:24 and one or more of SEQ ID NO:26, SEQ ID NO:27, or SEQ ID NO:28; or (iii) SEQ ID NO:25 and one or more of SEQ ID NO:26, SEQ ID NO:27, or SEQ ID NO:28; (b) capable of amplifying Leishmania braziliensis DNA having a nucleotide sequence of (i) SEQ ID NO: 16 and one or more of SEQ ID NO:19, SEQ ID NO:20, SEQ ID NO:21; (ii) SEQ ID NO:17 and one or more of SEQ ID NO:19, SEQ ID NO:20, or SEQ ID NO:21; or (iii) SEQ ID NO:18 and one or more of SEQ ID NO:19, SEQ ID NO:20, or SEQ ID NO:21; (c) capable of amplifying Leishmania DNA having a nucleotide sequence of (i) SEQ ID NO: 3 and one or more of SEQ ID NO:6, SEQ ID NO:7, or SEQ ID NO:8; (ii) SEQ ID NO:4 and one or more of SEQ ID NO:6, SEQ ID NO:7, or SEQ ID NO:8; (iii) SEQ ID NO:5 and one or more of SEQ ID NO:6, SEQ ID NO:7, or SEQ ID NO:8; (iv) SEQ ID NO:10 and one or more of SEQ ID NO:12, or SEQ ID NO:13; (v) SEQ ID NO:11 and one or more of SEQ ID NO:12, or SEQ ID NO:13; (d) capable of amplifying Leishmania chagasi DNA having a nucleotide sequence of (i) SEQ ID NO: 32 and one or more of SEQ ID NO:39, SEQ ID NO:40, SEQ ID NO:41, or SEQ ID NO:42; (ii) SEQ ID NO:33 and one or more of SEQ ID NO:39, SEQ ID NO:40, SEQ ID NO:41, or SEQ ID NO:42; (iii) SEQ ID NO:34 and one or more of SEQ ID NO:39, SEQ ID NO:40, SEQ ID NO:41, or SEQ ID NO:42; (iv) SEQ ID NO:35 and one or more of SEQ ID NO:39, SEQ ID NO:40, SEQ ID NO:41, or SEQ ID NO:42; (v) SEQ ID NO:36 and one or more of SEQ ID NO:39, SEQ ID NO:40, SEQ ID NO:41, or SEQ ID NO:42; (vi) SEQ ID NO:38 and one or more of SEQ ID NO:39, SEQ ID NO:40, SEQ ID NO:41, or SEQ ID NO:42; (e) capable of amplifying Trypanosoma cruzi DNA having nucleotide sequence of SEQ ID NO: 50 and SEQ ID NO:51; (f) capable of amplifying Trypanosoma cruzi DNA having a nucleotide sequence of SEQ ID NO: 53 and SEQ ID NO:54; (g) capable of amplifying Trypanosoma rangeli DNA having a nucleotide sequence of (i) SEQ ID NO:59 and one or more of SEQ ID NO: 56, SEQ ID NO:57, or SEQ ID NO:58; or (ii) SEQ ID NO:60 and one or more of SEQ ID NO:56, SEQ ID NO:57, or SEQ ID NO:58; (h) capable of amplifying Trypanosoma rangeli DNA having a nucleotide sequence of SEQ ID NO: 63 and SEQ ID NO:64; (i) capable of amplifying Leishmania major DNA having a nucleotide sequence of SEQ ID NO: 68 with one or more of SEQ ID NO: 66 or SEQ ID NO:67; (j) capable of amplifying Leishmania Mexicana or Leishmania amazonensis DNA having a nucleotide sequence of SEQ ID NO: 70 and SEQ ID NO:71; or (k) capable of amplifying Leishmania braziliensis, Leishmania panamensis, Leishmania guyanensis, or leishmania peruviana DNA having a nucleotide sequence of SEQ ID NO:75 and one or more of SEQ ID NO: 73 or SEQ ID NO:74. The nucleic acid probe can be an oligonucleotide having a nucleic acid sequence consisting of SEQ ID NO:9, 14, 77, 29, 30, 37, 52, 55, 61, 62, 65, 69, 72, or 76. In certain aspects the nucleic acid probe is labeled with a detectable label. The kit can further comprising amplification reagents. In further aspects the kit can further comprising reagents to detect a detectable label or a nucleic acid probe.

In a further embodiment a parasite detection kit can comprise at least one nucleic acid probe that is a detectably labeled oligonucleotide having a nucleic acid sequence consisting of SEQ ID NO:9, 14, 77, 29, 30, 37, 52, 55, 61, 62, 65, 69, 72, or 76.

Other embodiments are directed to methods for detecting a Leishmania or Trypanosoma parasite in a sample, comprising conducting a nucleic acid amplification reaction using one or more nucleic acid primer pairs as described herein and detecting the presence of parasite DNA by detecting primer pair specific amplicons. In certain aspects detecting primer pair specific amplicons comprises binding the amplified nucleic acid with a nucleic acid probe having a nucleic acid sequence consisting of SEQ ID NO:9, 14, 77, 29, 30, 37, 52, 55, 61, 62, 65, 69, 72, or 76, wherein binding of the probe to the amplified DNA indicates the presence of a parasite in the sample. The method can further comprising forming a reaction mixture comprising at least one primer pair and a sample portion prior to conducting the amplification reaction. In certain aspects the amplification reaction is an isothermal amplification reaction. In a further aspects the amplification reaction is a recombinase polymerase amplification (RPA). The biological sample can be a fluid or tissue sample. In certain aspects the fluid is blood, sputum, or saliva. In a further aspect the tissue sample is a tissue scrapping.

Other embodiments are directed to amplification devices comprising one or more primer pair described herein.

As used herein “biological sample” includes a fluid or tissue sample. Samples include sections of tissues such as biopsy and autopsy samples, and frozen sections taken for histologic purposes. In other aspects samples include blood and blood fractions or products (e.g., serum, plasma, platelets, red blood cells, and the like), saliva, sputum, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; bird; reptile; or fish, but also include insects.

A “target sequence” or “target nucleic acid sequence” as used herein means a nucleic acid sequence of a parasite as described herein, or a complement thereof, that is amplified, detected, or both amplified and detected using one or more of the polynucleotides herein provided. Additionally, while the term target sequence sometimes refers to a double stranded nucleic acid sequence, those skilled in the art will recognize that the target sequence can also be single stranded. In cases where the target is double stranded, polynucleotide primer sequences of the present invention preferably will amplify both strands of the target sequence. A target sequence may be selected that is more or less specific for a particular organism. For example, the target sequence may be specific to an entire genus, to more than one genus, to a species or subspecies, serogroup, serotype, strain, isolate or other subset of organisms. The polynucleotide sequences of the present invention are selected for their ability to specifically hybridize with a range of subspecies, or serotypes, of Trypanosomatida.

As used herein, the term “isolated” can refer to a nucleic acid or oligonucleotide that is substantially free of cellular material, bacterial material, viral material, or culture medium (when produced by recombinant DNA techniques) of their source of origin, or chemical precursors or other chemicals (when chemically synthesized). Moreover, an “isolated nucleic acid fragment” or “isolated oligonucleotide” is a nucleic acid or oligonucleotide that is not naturally occurring.

In certain aspects nucleic acid sequences are amplified before being detected. The term “amplified” defines the process of making multiple copies of the nucleic acid from a single or lower copy number of nucleic acid sequence molecule. The amplified nucleic acid can be referred to as an amplicon.

Moieties of the invention, such as oligonucleotides or amplicons may be labeled, that is conjugated or linked covalently or noncovalently to other moieties such as proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.” In certain aspects isolated nucleic acids or oligonucleotides are conjugated with a detectable label.

The term “labeled” with regard to a probe is intended to encompass direct labeling of the probe by coupling (i.e., physically linking) a detectable substance to the probe, as well as indirect labeling of the probe by reactivity with another reagent that is directly labeled. Examples of indirect labeling include detection of a probe using a fluorescently labeled antibody and end-labeling or centrally labeling of a DNA probe with biotin such that it can be detected with fluorescently labeled streptavidin.

A “label”, “detectable label”, or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

In certain aspects a fluorescent reporter dye, such as FAM dye (illustratively 6-carboxyfluorescein), is covalently linked to the 5′ end of an oligonucleotide probe. Other dyes illustratively include such TAMRA, AlexaFluor dyes such as AlexaFluor 495 or 590, Cascade Blue, Marina Blue, Pacific Blue, Oregon Green, Rhodamine, Fluoroscein, TET, HEX, Cy5, Cy3, and Tetramethylrhodamine. Each of the reporters can be quenched by a dye at the 3′ end or other non-fluorescent quencher. Quenching molecules are suitably matched to the fluorescence maximum of the dye. Any suitable fluorescent probe for use in nucleic acid amplification detection systems is illustratively operable in the instant invention. Similarly, any quenching molecule for use in such systems is illustratively operable.

The phrase “specifically binds” to a target refers to a binding reaction that is determinative of the presence of the molecule in the presence of a heterogeneous population of other biologics. Thus, under designated assay conditions, a specified molecule binds preferentially to a particular target and does not bind in a significant amount to other biologics present in the sample.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Specific amplification of different kinetoplastid parasites. Several set of primers were designed to expand a region inside the kinetoplast minicircle DNA to specifically amplify Leishmania infantum (also known as L. chagasi), Trypanosoma cruzi, and/or Leishmania braziliensis. The specificity of the amplification was confirmed by incubating the primers with different DNA templates, which included: the specific parasite DNA target (lane 1. L. infantum; lane 2. T. cruzi, lane 3. L. braziliensis), unrelated target DNA (lane 4. Human; lane 5. Dog; lane 6. Giardia) and primers without template (lane 7). Also, a 100 bp marker was included (lane 8).

FIG. 2. Sensitivity of the RPA tested on DNA purified from dog blood spiked with parasites. Heparinized dog blood was spiked with 1.3×10⁷ parasites and 1:4 serial dilutions were prepared. DNA was isolated from each dilution and resuspended in 100 μL of water. One μL of each dilution (which corresponds to, A. 130,000; B. 32,500; C. 8,125; D. 2,031; E. 508; F. 127; G. 32; H. 8; I. 2 parasites) was used as a template for RPA. Dilutions A-D showed strong amplification (not shown), and amplification was positive down to 2 parasites (I lane). Primers without template (J) and 100 bp marker (MK) are also shown.

FIG. 3. Basic RPA with Trypanosoma rangeli (panel). Primers: T. rangeli ITS1 FW, ITS1 RV. Samples—Water, T. rangeli, T. cruzi, L. braziliensis, L. chagasi, Human, Dog, and Giardia.

FIG. 4. Basic RPA with Leishmania major (panel). Primers: L. major 21 FW, 23 RV. Samples—Water, L. major, L. braziliensis, L. amazonensis, T. cruzi, Human, Dog, and Giardia.

FIG. 5. Basic RPA with Trypanosoma cruzi (panel). Primers: T. cruzi 11 FW, 11 RV. Samples—Water, Tc Meraloo, Tc Tejapileo, T. rangeli, L. braziliensis, L. amazonensis, L. chagasi, Human, Dog, and Giardia.

### DESCRIPTION

Protozoan parasites of the family Trypanosomatidae are the causative agent of devastating diseases in humans and livestock that range from self-curing cutaneous lesions to fatal systemic disease if not treated. Among the two genera, Leishmania and Trypanosoma, there are a multitude of species, each one resulting in a different disease manifestation. Many of the disease symptoms are common to other infections such as fever and malaise, making clinical diagnosis difficult. Correct diagnosis is essential because each agent responds to a different treatment. Thus, effective diagnosis is critical to treatment and control of these diseases.

Visceral leishmaniasis (VL), which is produced by L. infantum (=L. chagasi) (both in the New and Old World) or L. donovani (only in the Old World) accounts for 200-400,000 new cases each year, in addition to an undetermined proportion of subclinical infections and millions of people at risk (Alvar et al., 2012). VL is one of a group of “Neglected Tropical Diseases (NTDs)” identified by the World Health Organization as requiring major international efforts to reduce its negative impact on human kind.

Active VL is transmitted by the bite of infected sand flies of several different species. It is characterized by a progressive increase in visceral parasite burden, massive hepato-splenomegaly, anemia, cachexia, pancytopenia, and ultimately death if left untreated. A major constraint to diagnose visceral leishmaniasis is that the disease occurs in remote or socially depressed areas without basic health infrastructure or easy access to it. Therefore, in addition to low cost, features such as speed, sensitivity, and simplicity of the diagnostic method under field conditions are beneficial.

To tackle endemic diseases in resource-limited countries it is imperative to develop low-cost field applicable diagnostic tests. For this purpose, several isothermal amplification methods are being developed to detect pathogen DNA or RNA (Euler et al., 2013). In certain aspects the current methods use RPA isothermal method due to its comparative simplicity, high sensitivity and specificity, which makes it amenable to field application.

The RPA method described herein can be used as a low cost, sensitive, and field applicable diagnostic test to decrease or interrupt transmission of Leishmania (e.g., L. infantum) to human populations by improving the efficacy of current VL control programs of Public Health institutions, which are focused on early detection in humans and identification of infected dogs. Since dogs are the principal source of L. infantum infection to humans, the accurate identification of infected animals and subsequent removal from the transmission cycle is one of the most efficient strategies to decrease VL incidence in humans (Nunes et al, 2010). The importance of free-roaming dogs in L. infantum transmission was revealed during screenings carried in Posadas city, Argentina, at the beginning of an active VL control program. A higher infection prevalence was found in stray dogs (49%) collected by the Municipal Institute of Animal Health compared with 27% infection prevalence in dogs screened at households (L. Tartaglino, personal communication). A variety of serological methods (ELISA, DAT, IFAT, K39) have been used to identify infected dogs, yet an ill-defined proportion of seronegative dogs could still be infected with L. infantum and representing a risk to humans, as determined by molecular methods (Mohammadiha et al., 2013). In some cases up to 70% of asymptomatic dogs are not detected by serological methods.

In certain embodiments, the compositions and methods described herein can be used to accurately detect Leishmania (e.g., L. infantum) infections in endemic areas where other Leishmania species (L. braziliensis) and Trypanosoma cruzi co-exist using isothermal amplification, e.g., RPA. In addition to L. infantum, the inventors have designed primers that comprise other trypanosomatids that could overlap in some endemic areas, i.e., L. braziliensis and T. cruzi. In certain aspects the methods described herein can be used as multiplex or for single-parasite diagnosis.

Various aspects include one or more of (a) RPA procedures and conditions for detecting Leishmania, (b) DNA extraction methods suitable for point of care implementation, (c) species-specific dual labeled probes to detect the parasites in single or multiplex lateral flow formats, and (d) validation of the assays using samples obtained from areas endemic for these parasites.

### I. Detecting Parasites

RPA was developed to conduct isothermal amplification in the field using a mini (portable) extractor to obtain DNA from blood or serum at the point of care. In certain aspects RPA can be conducted using a sample of blood spotted on a filter paper, dried, and then boiled in water to release the DNA. The use of lateral flow to detect DNA amplification is based on dual labeled probes and gold nanoparticles. This approach allows multiplex detection in the same samples. In certain aspects the method is used to discriminate trypanosomatid infections at the point of care, a concept that can be applied to other groups of co-existing pathogens.

RPA to detect Leishmania as a proof of concept: to develop the RPA inventors selected the kinetoplast DNA minicircles as targets for designing the corresponding primers due to the high copy number of this structure (mitochondrion).

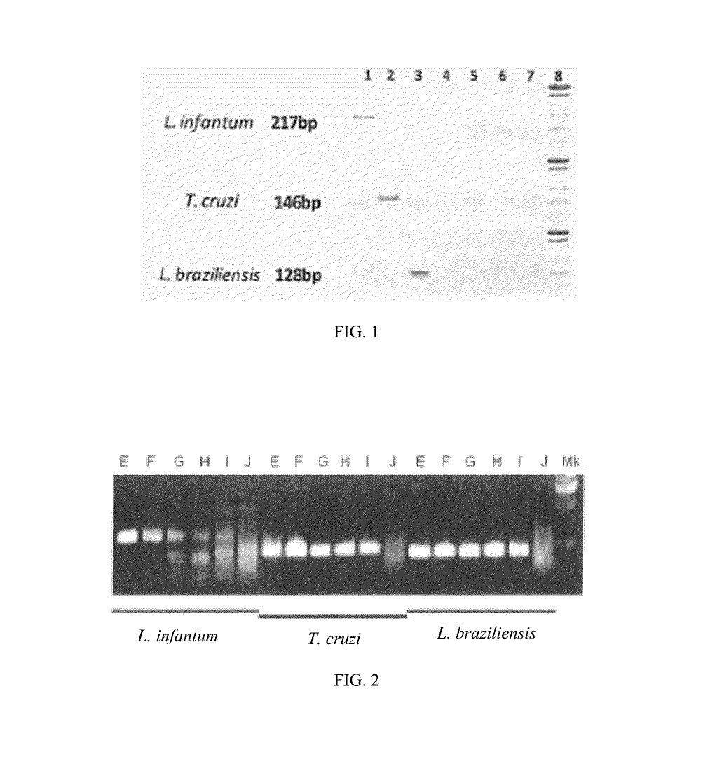

Results showed that the RPA was capable of distinguishing between L. infantum, L. braziliensis, and T. cruzi (FIG. 1). Furthermore, the inventors have shown that RPA can detect as few as 2 parasites upon amplification of different numbers of L. infantum from which DNA was extracted (under POC-like conditions) from blood (FIG. 2).

Primer sets were designed. Primers were typically 30-35 nucleotides long, for each target species followed by alignments between all sequences reported in GenBank for minicircle kDNA using the Clustal W program. Primers were designed in a target region with 40-60% GC content, few direct/inverted repeats and absence of long homo-polymer tracks. The inventors focused principally on conserved regions and to a lesser extent on regions with moderate variability, obtaining RPA amplicons between 220 bp and 120 bp as detected in agarose gels (FIG. 1).

Recombinase Polymerase Amplification (RPA) uses a recombinase to specifically pair primers with double-stranded DNA on the basis of homology, thus directing DNA synthesis from defined DNA sequences present in the sample. Presence of the target sequence initiates DNA amplification, and no thermal or chemical melting of DNA is required. The reaction progresses rapidly and results in specific DNA amplification from just a few target copies to detectable levels typically within 5-10 minutes. The entire reaction system is stable as a dried formulation and does not need refrigeration. RPA can be performed in a 10, 20, 30, 40, 50, 60, 70, 80, 90, 100 μl or more volume. The reaction can contain at least two amplification primers at 50, 100, 150, 200, 250, 300, 350, 400, 450 nM each, or any value or range there between. In certain aspect the reaction contains a tagged RPA probe. The reaction solution is buffer with appropriate agent and includes any metal or other additives required for enzyme activity. Reagents can be prepared as a master mix. Template nucleic acids or sample DNA can be added to the master mix that had been distributed into reaction tubes containing a dried enzyme pellet. In certain aspects Mg acetate can be pipetted into the tube lids. To start the reaction the lids are closed, and the Mg acetate is centrifuged into the tubes using a minispin centrifuge, and the tubes are immediately placed into a tube scanner or lateral flow device. The reaction is incubated a temperature from 25, 30, 35, 40, 45, to 50° C., including all values and ranges there between. Certain embodiments are directed kits or ready to use tubes or devices (e.g., lateral flow devices) comprising the appropriate primers and/probes.

Lateral flow tests or Lateral Flow Assays are simple devices intended to detect the presence or absence of a target analyte in sample without the need for specialized equipment. Lateral flow assay can be used for point of care testing. Lateral flow assays are based on a capillary bed, such as pieces of porous paper, sintered polymer or the like. The capillary bed has the capacity to transport fluid spontaneously. In certain aspects a sample pad can act as a sponge and holds an excess of sample fluid. Once soaked, the fluid migrates to a second element (conjugate pad) in which the manufacturer has stored the so-called conjugate, a dried format of bio-active particles or probes in a matrix that contains everything to guarantee an optimized reaction between the target molecule and its binding partner that can be immobilized on or to a surface. While the sample fluid dissolves the matrix, it also dissolves the particles and in one combined transport action the sample and conjugate mix while flowing through the porous structure. In this way, the analyte binds to the particles while migrating further through an additional capillary bed. This material has one or more areas (often called stripes) where a third molecule has been immobilized. By the time the sample-conjugate mix reaches these strips, analyte has been bound on the particle and the third ‘capture’ molecule binds the complex. After a while, when more and more fluid has passed the stripes, particles accumulate and the area changes color. After passing these reaction zones the fluid enters the final porous material, the wick, that simply acts as a waste container.

Primer design for Leishmania chagasi (GenBank JX156627.1 (gi 394857980); SEQ ID NO:1). Primers include: Lcha FW1, GATCGAGGCTGACACAATTTGATGGCCTGTG (SEQ ID NO:3), 31nt, GC % 51.6, TM 64, Hg, 3, SF 20; Lcha FW1.1—TGGCCTGTGTAAATATATCTTACCTTAAGGTGGC (SEQ ID NO:4), 34nt, GC % 41.2, TM 60.7, Hg 1, SF20; Lcha FW3—TCTGTTTGGTTATGTTATGATCGAGGCTGAC (SEQ ID NO:5), 31nt, Region NCR, GC % 41.9, TM 59.8, Hg 6, SF 16; Lcha RV2—CTACCCGGAGGACCAGAAAAGTTTGGGA (SEQ ID NO:6), 28 nt, region CR, GC % 53.6, TM 63.2; Lcha RV4—TTCGGGATTTTCCACCAATTCCCAACCATC (SEQ ID NO:7), 30 nt, GC % 46.7, TM 62.5; Lcha RV4.5—ACCAATTCCCAACCATCCAACACGTCCCAA (SEQ ID NO:8), 30 nt, GC % 50, TM 65.1, Hg 4, SF 9. (Hg=Hairpin, SF=selfdimer)

Amplicon sizes of various L. infantum (=L. chagasi) primer combinations are provided below.

| | Primer Combination | |
|---|---|---|
| Samples | (L. chagasi) | Amplicon (bp) |
| 1/2/12 | Fw1/RV2 | 199 |
| 3/4/34 | Fw1/RV4 | 148 |
| 5/6/56 | Fw1/RV4.5 | 135 |
| 7/8/78 | Fw1.1/RV2 | 177 |
| 9/10/910 | Fw1.1/RV4 | 126 |
| 11/12/1112 | Fw1.1/RV4.5 | 113 |
| 13/14/1314 | Fw3/RV2 | 217 |
| 15/16/1516 | Fw3RV4 | 166 |
| 17/18/1718 | Fw3/RV4.5 | 153 |
| 19/20/1920 | Lbra20/Lbra40 | 128 |

Primers for Leishmania braziliensis kinetoplast minicircle (gi|643458|gb|U19803.1; SEQ ID NO:15). Primers include: Lbra 10-FW—TGCGCAGAGTTATTATGTTGTCAATGAATAA (SEQ ID NO:16), 31nt, GC % 32.3, TM 57.2, Hg 10, SF 25; Lbra FW20—GATGAAAATGTACTCCCCGACATGCCTCTG (SEQ ID NO:17), 30nt, GC % 50, TM 62, Hg 2, SF 17; Lbra FW25—AATTTGATGAAAATGTACTCCCCGACATGC (SEQ ID NO:18), 30nt, GC % 40, TM 59.4, Hg 3, SF 20; Lbra RV30—TGAGACGGGGTTTCTGTATGCCATTTTTCGGT (SEQ ID NO:19), 31nt, GC % 45.2, TM 63.4, Hg 1, SF 18; Lbra RV40—CTAATTGTGCACGGGGAGGCCAAAAATAGCGA (SEQ ID NO:20), 32nt, GC % 50, TM 64.9, Hg 2, SF 20; Lbra RV45—AACCCCTAATTGTGCACGGGGAGGCCAAA (SEQ ID NO:21), 29nt, GC % 55.2, TM 67.1, Hg 1, SF 19.

| | Primer Combination | |
|---|---|---|
| Samples | (L. braziliensis) | Amplicon (bp) |
| 40/41/41 | FW10/RV30 | 136 |
| 43/44/45 | FW10/RV40 | 183 |
| 46/47/48 | FW10/RV45 | 188 |
| 49/50/51 | FW20/RV30 | 82 |
| 52/53/54 | FW20RV40 | 128 |
| 55/56/57 | FW20Rv45 | 133 |
| 58/59/60 | FW25/RV30 | 86 |
| 61/62/63 | FW25/RV40 | 133 |
| 64/65/66 | FW25RV45 | 138 |
| 67/68/69 | Lcha FW1.1/RV4.5 | 113 |
| 70/71/72 | Lcha 100FW/110RV | |

Trypanosoma cruzi Y strain kinetoplast minicircle sequence, clone M835, orf, complete cds >gi|473252|gb|U07845.1|TCU07845 (SEQ ID NO:22). Primers include: T. cruzi-60-FW—AGCCTTATCACCCTTACCACCCACAGCCTA (SEQ ID NO:23), 30nt, GC % 53.3, TM 65.2, Hg 2, SF14; Tcruzi-65-FW—CAGCCTATATTACACCAACCCCAATCGAAC (SEQ ID NO:24), 30nt, GC % 46.7, TM 60.5, Hg 2, SF 15; T.cruzi-70-FW—ACCCTTACCACCCACAGCCTATATTACACCA (SEQ ID NO:25), 31nt, GC % 48.4, TM 63.3, Hg 3, SF14; Tcruzi-80-RV—GCGTTCAACTTTTGGGGCCCAGAATCATGC (SEQ ID NO:26), 30nt, GC % 53.3, 65.1, Hg 4, SF19; T. cruzi-85-RV—AACTTTTGGGGCCCAGAATCATGCATCTCC (SEQ ID NO:27), 30nt, GC % 50, TM 64, Hg 4, SF20; T. cruzi-90-RV—CTGCAAGAAACTGGAAAATATGGTTTTGGGAG (SEQ ID NO:28), 32nt, GC % 40.6, TM 59.9, Hg 2, SF19.

| | Primer Combination | |
|---|---|---|
| Samples | (T. cruzi) | Amplicon (bp) |
| A/B/C | FW60/RV80 | 135 |
| D/E/F | FW60/RV85 | 129 |
| G/H/I | FW60/RV90 | 169 |
| J/K/L | FW65/RV80 | 112 |
| M/N/O | FW65RV85 | 106 |
| P/Q/R | FW65RV90 | 146 |
| S/T/U | FW70/RV80 | 126 |
| V/W/X | FW70RV/85 | 120 |
| Y/Z/CH | FW70RV/90 | 160 |
| 80/81/82 | LchaFW1.1/RV4.5 | 113 |
| 83/84/85 | Lbra FW20/RV40 | 128 |
| 86/87/88 | Lbra FW25/RV40 | 133 |

gi|4761805|gb|AF103738.1| Leishmania chagasi kinetoplast minicircle DNA, complete sequence (SEQ ID NO:31). Primers include: LDOCHIN-FW100—TAGCCATAGCGCTTTAGAATAGTTCGRCTCCGAA (SEQ ID NO:32), 34nt, GC 45.6 Tm 63.5; LDOCHIN-FW110—TATAGCCATAGCGCTTTAGAATAGTTCGRCTCCGA (SEQ ID NO:33), 35nt, GC 44.3, TM 62.9; LDOCHIN-FW120—TATAGCCATAGCGCTTTAGAATAGTTCG (SEQ ID NO:34), 28nt, GC 39.3, TM 56.3; LDOCHIN-FW130—TRCTCGTACACTATAAGTATTATGTTTAATATAT (SEQ ID NO:35), 34nt, GC 22.1, TM 52.1; LDOCHIN-FW140—CTGACATTRCTCGTACACTATAGTATTATG (SEQ ID NO:36), 31nt, GC 33.9, TM 54.2; TAATAACTFACATTACTCGTACACTATAAGTATTATGTTTAATAT (SEQ ID NO:37); LDOCHIN-FW150—TAGTAATATCTRTACCGATATATTTRTAGGTTG (SEQ ID NO:38); LDOCHIN-RV160—CAACCTAYAAATATATCGGTAYAGATATTACTA (SEQ ID NO:39), 33nt, GC 27.3, TM 52.6; LDOCHIN-FW170—CATACTGCAGTGAATTGRAAATTAATRAATTGG (SEQ ID NO:40), 33nt, GC 30.3 TM 55.9; LDOCHIN-RV180—CCAATTYATTAATTTYCAATTCACTGCAGTATG (SEQ ID NO:41); LDOCHIN-RV190—CCCTACCCGGAGGACCAGAAAAGTTTGG (SEQ ID NO:42), 28nt, GC 57.1, TM 63.9; Probe CCAAACTTTTCTGGTCCTCCGGGTAGGGGCGTTCTGCAAA (SEQ ID NO:43).

Other primers include GAAAAATGGGTCCAGAAATGCCGTTCAA (SEQ ID NO:44); TTGAACGGGATTTCTGCACCCATTTTTC (SEQ ID NO:45); AAACTGGGGGTTGGTGTAAAATAGGGCCGG (SEQ ID NO:46); CCGGCCCTATTTTACACCAACCCCCAGTTT (SEQ ID NO:47); GGAAACTGGGGGTTGGTGTAAAATAGGGC (SEQ ID NO:48); and GCCCTATTTTACACCAACCCCCAGTTTCC (SEQ ID NO:49)

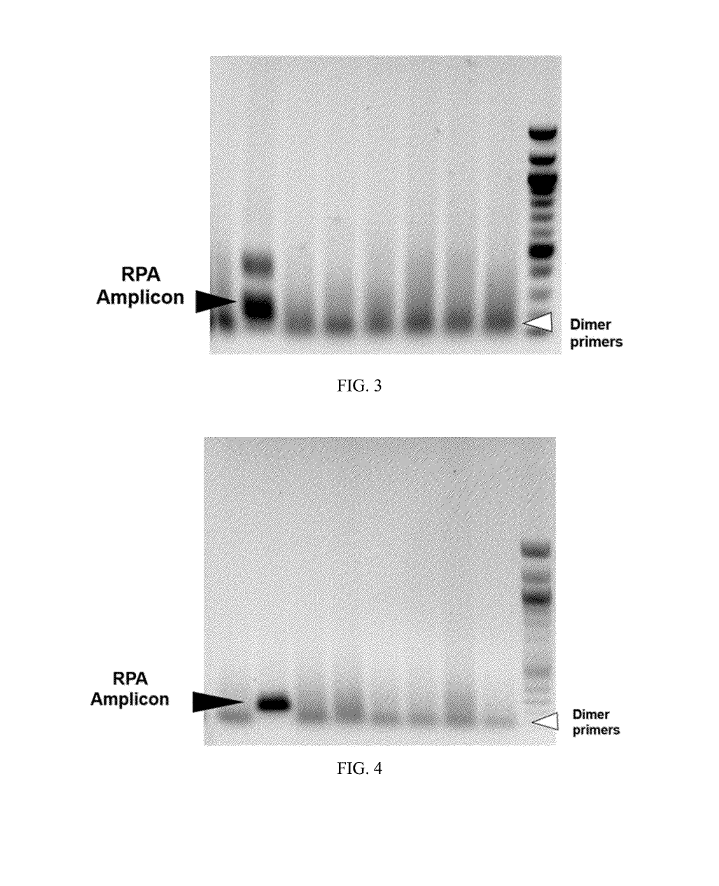

Basic RPA with Trypanosoma rangeli (FIG. 3). Primers: T. rangeli ITS1 FW, ITS1 RV. Samples—Water, T. rangeli, T. cruzi, L. braziliensis, L. chagasi, Human, Dog, Giardia. Reagents were combined using sterile techniques (mixing in template samples on a different bench after the use of the other reagents, interacting with negative samples before positive, etc.). The sample solutions were then amplified using RPA and were incubated in 42 degrees Celsius for 40 minutes. They were run on a 1% agarose gel stained with ethidium bromide at 120 V for approximately 50 minutes. Results include: The T. rangeli primers only amplified the T. rangeli target. No cross-reaction was seen between the T. rangeli primers and any of the other samples.

Basic RPA with Leishmania major (FIG. 4). Primers: L. major 21 FW, 23 RV. Samples—Water, L. major, L. braziliensis, L. amazonensis, T. cruzi, Human, Dog, and Giardia. Reagents were combined using sterile techniques (mixing in template samples on a different bench after the use of the other reagents, interacting with negative samples before positive, etc.). The sample solutions were then amplified using RPA and were incubated in 42 degrees Celsius for 40 minutes. They were run on a 1% agarose gel stained with ethidium bromide at 120 V for approximately 50 minutes. Results: L. major primers were successful in amplifying the L. major target. No cross-reaction was seen between the L. major primers and any of the other samples.

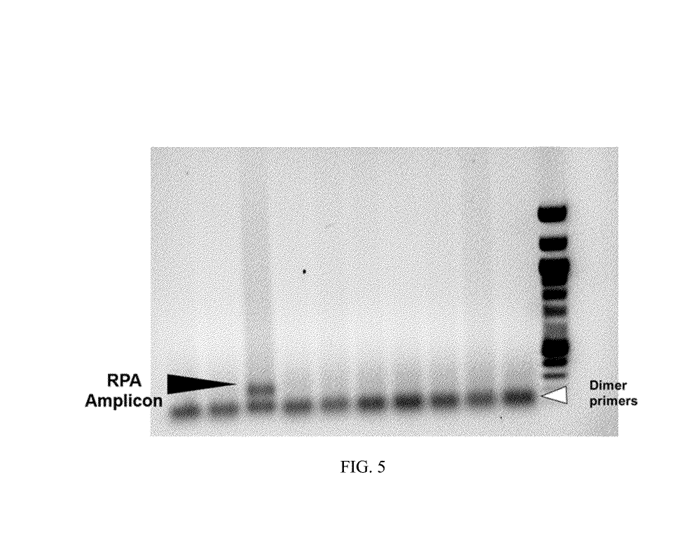

Basic RPA with Trypanosoma cruzi (FIG. 5). Primers: T. cruzi 11 FW, 11 RV. Samples—Water, Trypanosoma cruzi Tc Meraloo, Trypanosoma cruzi Tc Tejapileo, T. rangeli, L. brazdiensis, L. amazonensis, L. chagasi, Human blood, Dog blood, and Giardia. Reagents were combined using sterile techniques (mixing in template samples on a different bench after the use of the other reagents, interacting with negative samples before positive, etc.). The sample solutions were then amplified using RPA and were incubated in 42 degrees Celsius for 40 minutes. They were run on a 1% agarose gel stained with ethidium bromide at 120 V for approximately 50 minutes. Results: The T. cruzi primers seem to have been successful in amplifying only the Tc Tejapileo sample and not the Tc Meraloo. The T. cruzi specific primers did not cross-react with any of the other samples.

### II. Leishmania Treatments

Specific antileishmanial therapy is not routinely indicated for uncomplicated LCL caused by strains that have a high rate of spontaneous resolution and self-healing (L. major, L. mexicana). Lesions that are extensive, severely inflamed, or located where a scar would result in disability (near a joint) or cosmetic disfigurement (face or ear), that involve the lymphatics, or that do not begin healing within 3-4 mo should be treated. Cutaneous lesions suspected or known to be caused by members of the Viannia subgenus (New World) should be treated because of the low rate of spontaneous healing and the potential risk for development of mucosal disease. Similarly, patients with lesions caused by L. tropica (Old World), which are typically chronic and nonhealing, should be treated. All patients with VL or ML should receive therapy.

The pentavalent antimony compounds (sodium stibogluconate [Pentostam, GlaxoSmithKline, Uxbridge, UK] and meglumine antimoniate [Glucantime, Aventis, Strasbourg, France]) have been the mainstay of antileishmanial chemotherapy for >40 yr. These drugs have similar efficacies, toxicities, and treatment regimens. Currently, for sodium stibogluconate (available in the USA from the Centers for Disease Control and Prevention, Atlanta, Ga.), the recommended regimen is 20 mg/kg/day intravenously or intramuscularly for 20 days (for LCL and DCL) or 28 days (for ML and VL). Repeated courses of therapy may be necessary in patients with severe cutaneous lesions, ML, or VL. An initial clinical response to therapy usually occurs in the 1st wk of therapy, but complete clinical healing (re-epithelialization and scarring for LCL and ML, and regression of splenomegaly and normalization of cytopenias for VL) is usually not evident for weeks to a few months after completion of therapy. Cure rates with this regimen of 90-100% for LCL, 50-70% for ML, and 80-100% for VL were common in the 1990s, but treatment failures, especially in children, have become common in parts of India, East Africa, and Latin America. Relapses are common in patients who do not have an effective antileishmanial cellular immune response (DCL or HIV co-infection). Adverse effects of antimony therapy are dose and duration dependent and commonly include fatigue, arthralgias and myalgias (50%), abdominal discomfort (30%), elevated hepatic transaminase level (30-80%), elevated amylase and lipase levels (almost 100%), mild hematologic changes (slightly decreased leukocyte count, hemoglobin level, and platelet count) (10-30%), and nonspecific T-wave changes on electrocardiography (30%). Sudden death due to cardiac toxicity has rarely been reported with use of very high doses of pentavalent antimony.

Amphotericin B desoxycholate and the amphotericin lipid formulations are very useful in the treatment of VL or ML and in some regions have replaced antimony as first-line therapy. However, the prohibitively high cost of these drugs precludes their use in many resource-poor regions of the world. Amphotericin B desoxycholate at doses of 0.5-1.0 mg/kg every day or every other day for 14-20 doses achieved a cure rate for VL of close to 100%, but the renal toxicity commonly associated with amphotericin B was common. The lipid formulations of amphotericin B are especially attractive for treatment of leishmaniasis because the drugs are concentrated in the reticuloendothelial system and are less nephrotoxic. Liposomal amphotericin B is highly effective, with a 90-100% cure rate for VL in immunocompetent children, some of whom were refractory to antimony therapy. Liposomal amphotericin B (Ambisome, Gilead Sciences, Foster City, Calif.) is approved by the U.S. Food and Drug Administration for treatment of VL at a recommended dose for immunocompetent patients of 3 mg/kg on days 1-5, 14, and 21 and should be considered for first-line therapy in the USA. Therapy for immunocompromised patients may need to be prolonged. A single high dose of liposomal amphotericin B (10 mg/kg) was found to be non-inferior to conventional amphotericin (15 doses of 1 mg/kg) in India and offers a less cost-prohibitive approach. Parenteral treatment of VL with the aminoglycoside paromomycin (aminosidine) has efficacy (˜95%) similar to that of amphotericin B in India. Miltefosine, a membrane-activating alkylphospholipid, has been recently developed as the 1st oral treatment for VL and has a cure rate of 80-90% in Indian patients with VL when administered orally at 50-100 mg/day (or 2.5 mg/kg for children aged <12 years) for 28 days. Gastrointestinal adverse effects were frequent but did not require discontinuation of the drug. An increased rate of relapse (up to 20%) has been seen in children treated with miltefosine. Dose-sparing combination regimens are being actively investigated for treatment of VL. Treatment of LCL with oral drugs has had only modest success. Ketoconazole has been effective in treating adults with LCL caused by L. major, L. mexicana, and L. panamensis but not L. tropica or L. braziliensis. Fluconazole in high doses (up to 8 mg/kg/d) for 4-8 wks was demonstrated to be effective in treating LCL in studies in both the Old and New World, however, the experience in young children is limited. Miltefosine 2.5 mg/kg/day orally for 20-28 days was effective in 70-90% of patients with LCL in the Americas. Topical treatment of LCL with paromomycin ointment has been effective in selected areas in the both Old and New World. Enhanced drug development efforts and clinical trials of new drugs are clearly needed, especially in children.

1. A parasite detection kit comprising at least one of:
(A) a nucleic acid amplification primer pair:
(a) capable of amplifying Trypanosome cruzi DNA having a nucleotide sequence of (i) SEQ ID NO: 23 and one or more of SEQ ID NO:26, SEQ ID NO:27, or SEQ ID NO:28; (ii) SEQ ID NO:24 and one or more of SEQ ID NO:26, SEQ ID NO:27, or SEQ ID NO:28; or (iii) SEQ ID NO:25 and one or more of SEQ ID NO:26, SEQ ID NO:27, or SEQ ID NO:28;
(b) capable of amplifying Leishmania braziliensis DNA having a nucleotide sequence of (i) SEQ ID NO: 16 and one or more of SEQ ID NO:19, SEQ ID NO:20, SEQ ID NO:21; (ii) SEQ ID NO:17 and one or more of SEQ ID NO:19, SEQ ID NO:20, or SEQ ID NO:21; or (iii) SEQ ID NO:18 and one or more of SEQ ID NO:19, SEQ ID NO:20, or SEQ ID NO:21;
(c) capable of amplifying Leishmania DNA having a nucleotide sequence of (i) SEQ ID NO: 3 and one or more of SEQ ID NO:6, SEQ ID NO:7, or SEQ ID NO:8; (ii) SEQ ID NO:4 and one or more of SEQ ID NO:6, SEQ ID NO:7, or SEQ ID NO:8; (iii) SEQ ID NO:5 and one or more of SEQ ID NO:6, SEQ ID NO:7, or SEQ ID NO:8; (iv) SEQ ID NO:10 and one or more of SEQ ID NO:12, or SEQ ID NO:13; (v) SEQ ID NO:11 and one or more of SEQ ID NO:12, or SEQ ID NO:13;
(d) capable of amplifying Leishmania chagasi DNA having a nucleotide sequence of (i) SEQ ID NO: 32 and one or more of SEQ ID NO:39, SEQ ID NO:40, SEQ ID NO:41, or SEQ ID NO:42; (ii) SEQ ID NO:33 and one or more of SEQ ID NO:39, SEQ ID NO:40, SEQ ID NO:41, or SEQ ID NO:42; (iii) SEQ ID NO:34 and one or more of SEQ ID NO:39, SEQ ID NO:40, SEQ ID NO:41, or SEQ ID NO:42; (iv) SEQ ID NO:35 and one or more of SEQ ID NO:39, SEQ ID NO:40, SEQ ID NO:41, or SEQ ID NO:42; (v) SEQ ID NO:36 and one or more of SEQ ID NO:39, SEQ ID NO:40, SEQ ID NO:41, or SEQ ID NO:42; (vi) SEQ ID NO:38 and one or more of SEQ ID NO:39, SEQ ID NO:40, SEQ ID NO:41, or SEQ ID NO:42;
(e) capable of amplifying Trypanosoma cruzi DNA having nucleotide sequence of SEQ ID NO: 50 and SEQ ID NO:51;
(f) capable of amplifying Trypanosoma cruzi DNA having a nucleotide sequence of SEQ ID NO: 53 and SEQ ID NO:54;
(g) capable of amplifying Trypanosoma rangeli DNA having a nucleotide sequence of (i) SEQ ID NO:59 and one or more of SEQ ID NO: 56, SEQ ID NO:57, or SEQ ID NO:58; or (ii) SEQ ID NO:60 and one or more of SEQ ID NO:56, SEQ ID NO:57, or SEQ ID NO:58;
(h) capable of amplifying Trypanosoma rangeli DNA having a nucleotide sequence of SEQ ID NO: 63 and SEQ ID NO:64;
(i) capable of amplifying Leishmania major DNA having a nucleotide sequence of SEQ ID NO: 68 with one or more of SEQ ID NO: 66 or SEQ ID NO:67;
(j) capable of amplifying Leishmania Mexicana or Leishmania amazonensis DNA having a nucleotide sequence of SEQ ID NO: 70 and SEQ ID NO:71; or
(k) capable of amplifying Leishmania braziliensis, Leishmania panamensis, Leishmania guyanensis, or leishmania peruviana DNA having a nucleotide sequence of SEQ ID NO:75 and one or more of SEQ ID NO: 73 or SEQ ID NO:74; and
(B) a nucleic acid probe for detecting amplified Leishmania or Trypanosoma DNA segments.

2. The kit of claim 1, wherein the nucleic acid probe is an oligonucleotide having a nucleic acid sequence consisting of SEQ ID NO:9, 14, 77, 29, 30, 37, 52, 55, 61, 62, 65, 69, 72, or 76.

3. The kit of claim 2, wherein the nucleic acid probe is labeled with a detectable label.

4. The kit of claim 1, further comprising amplification reagents.

5. The kit of claim 1, further comprising one or more of a recombinase enzyme, a single-stranded DNA binding protein, or strand-displacing polymerase.

6. The kit of claim 1, further comprising an exonuclease.

7. The kit of claim 6, wherein the exonuclease is an exonuclease III.

8. The kit of claim 1, further comprising an endonuclease.

9. The kit of claim 8, wherein the endonuclease is an endonuclease IV.

10. The kit of claim 1, further comprising reagents to detect a detectable label.

11. A parasite detection kit comprising at least one nucleic acid probe that is a detectably labeled oligonucleotide having a nucleic acid sequence consisting of SEQ ID NO:9, 14, 77, 29, 30, 37, 52, 55, 61, 62, 65, 69, 72, or 76.

12. A method for detecting a Leishmania or Trypanosoma parasite in a sample, comprising conducting a nucleic acid amplification reaction using one or more nucleic acid primer pairs of claim 1 and detecting the presence of parasite DNA by detecting primer pair specific amplicons.

13. The method of claim 12, wherein detecting primer pair specific amplicons comprises binding the amplified nucleic acid with a nucleic acid probe having a nucleic acid sequence consisting of SEQ ID NO:9, 14, 77, 29, 30, 37, 52, 55, 61, 62, 65, 69, 72, or 76, wherein binding of the probe to the amplified DNA indicates the presence of a parasite in the sample.

14. The method of claim 12, further comprising forming a reaction mixture comprising at least one primer pair and a sample prior to conducting the amplification reaction.

15. The method of claim 12, wherein the amplification reaction is an isothermal amplification reaction.

16. The method of claim 12, wherein the amplification reaction is a recombinase polymerase amplification (RPA).

17. The method of claim 12, wherein the biological sample is fluid or tissue sample.

18. The method of claim 17, wherein the fluid is blood, sputum, or saliva.

19. The method of claim 17, wherein the tissue sample is a tissue scrapping.

20. An amplification device comprising one or more primer pair of claim 1.
